(19) United States
(12) Patent Application Publication
(10) Pub. No.: US 2013/0204137 A1
(43) Pub. Date: Aug. 8, 2013

(54) Method and System for Denoising Acoustic Travel Times and Imaging a Volume of Tissue

(71) Applicant: DELPHINUS MEDICAL TECHNOLOGIES, INC., Plymouth, MI (US)

(72) Inventors: Olivier Roy, Royal Oak, MI (US); Cuiping Li, Troy, MI (US); Nebojsa Duric, Bloomfield Hills, MI (US)

(73) Assignee: DELPHINUS MEDICAL TECHNOLOGIES, INC., Plymouth, MI (US)

(21) Appl. No.: 13/756,864

(22) Filed: Feb. 1, 2013

Related U.S. Application Data
(60) Provisional application No. 61/636,827, filed on Apr. 23, 2012.
(60) Provisional application No. 61/594,879, filed on Feb. 3, 2012.

(51) Int. Cl. A61B 8/15

(52) U.S. Cl. CPC A61B 8/15
USPC 600/448

(57) ABSTRACT

A method and system for denoising acoustic travel times and imaging a volume of tissue comprising receiving a dataset representative of acoustic waveforms originating from an array of ultrasound emitters and received with an array of ultrasound receivers; for each ultrasound emitter in the array of ultrasound emitters, forming an empirical relative travel time matrix, from the dataset, including a set of relative empirical travel times, each relative empirical travel time corresponding to a pair of ultrasound receivers receiving an acoustic waveform, generating a denoised empirical relative travel time matrix, and extracting a set of denoised absolute travel times from the denoised empirical relative travel time matrix; and rendering an image of the volume of tissue based on an acoustomechanical parameter and the set of denoised absolute travel times corresponding to each ultrasound emitter in the array of ultrasound emitters.

### CROSS-REFERENCE TO RELATED APPLICATIONS

This application claims the benefit of U.S. Provisional Application Ser. No. 61/636,827, filed on 23-Apr.-2012, and U.S. Provisional Application Ser. No. 61/594,879, filed on 3-Feb.-2012, which are incorporated in their entirety by this reference.

### TECHNICAL FIELD

This invention relates generally to the medical imaging field, and more specifically to an improved method and system for denoising acoustic travel times and imaging a volume of tissue.

### BACKGROUND

Time delay estimation plays a role in a large number of applications, including ultrasound tomography and array calibration. In ultrasound travel time tomography, the speed of sound can be imaged based on travel time data measured using a transducer array surrounding the propagation medium of interest (e.g., tissue). When ultrasound tomography is applied to breast imaging, the sound speed image can provide valuable information to detect cancer in tissues at an early stage. For such applications, accurate acoustic travel time estimation (e.g., travel time of a signal from an ultrasound emitter to an ultrasound receiver) can be used to provide images that are free of artifacts and that display accurate sound speed values in the sound speed image.

Although several travel time estimation methods have been developed, accurate travel time estimation remains a challenging task in practice. Cross-talk among nearby transducers, non-ideal frequency response of piezoelectric sensors, and strong attenuation in the propagation medium of interest are some of the reasons that the ultrasound signals under observation are “noisy” and otherwise distorted, thereby making accurate travel time estimation more difficult.

Thus, there is a need in the medical imaging field to create an improved method and system for denoising acoustic travel times and imaging a volume of tissue. This invention provides such a method and system.

### BRIEF DESCRIPTION OF THE FIGURES

FIG. 1 is a schematic of a transducer array depicting a tomographic setup;

FIG. 2 is a flowchart depicting an embodiment of a method for denoising acoustic travel times and imaging a volume of tissue;

FIGS. 3-4 are flowcharts depicting a first embodiment of generating a denoised empirical relative travel time matrix;

FIG. 5 is a flowchart depicting another embodiment of a method for denoising acoustic travel times and imaging a volume of tissue;

FIGS. 6 and 7 are flowcharts depicting a third and a fourth embodiment of generating a denoised empirical relative travel time matrix, respectively;

FIGS. 8A-8C and 9 are schematics of the system for imaging a volume of tissue of a preferred embodiment;

FIG. 10 is a plot showing data derived from illustrative examples of the methods for denoising acoustic travel times of preferred embodiments; and

FIGS. 11A-11D are acoustic speed image representations based on data derived from illustrative examples of the system and method for denoising acoustic travel times of preferred embodiments.

### DESCRIPTION OF THE PREFERRED EMBODIMENTS

The following description of preferred embodiments of the invention is not intended to limit the invention to these preferred embodiments, but rather to enable any person skilled in the art to make and use this invention.

### 1. Optimization Theory and Technique for Denoising Acoustic Travel Times

The method 100 for denoising acoustic travel times is preferably used to obtain denoised acoustic travel times for acoustic waveforms interacting with a volume of tissue (e.g., interaction can include acoustic reflection, acoustic transmission, and acoustic attenuation). The denoised acoustic travel times are preferably used to generate an acoustic speed, an acoustic reflection, and/or an acoustic attenuation image rendering of a volume of tissue scanned by ultrasound emitters and ultrasound receivers surrounding tissue. Use of the denoised acoustic travel times results in image renderings that have fewer artifacts and more accurate acoustic speed values, thereby leading to more a clearer and more accurate depiction of the scanned volume of tissue. The method 100 is preferably used independently to denoise acoustic travel times, but can alternatively be applied subsequently to any suitable acoustic travel time estimation method.

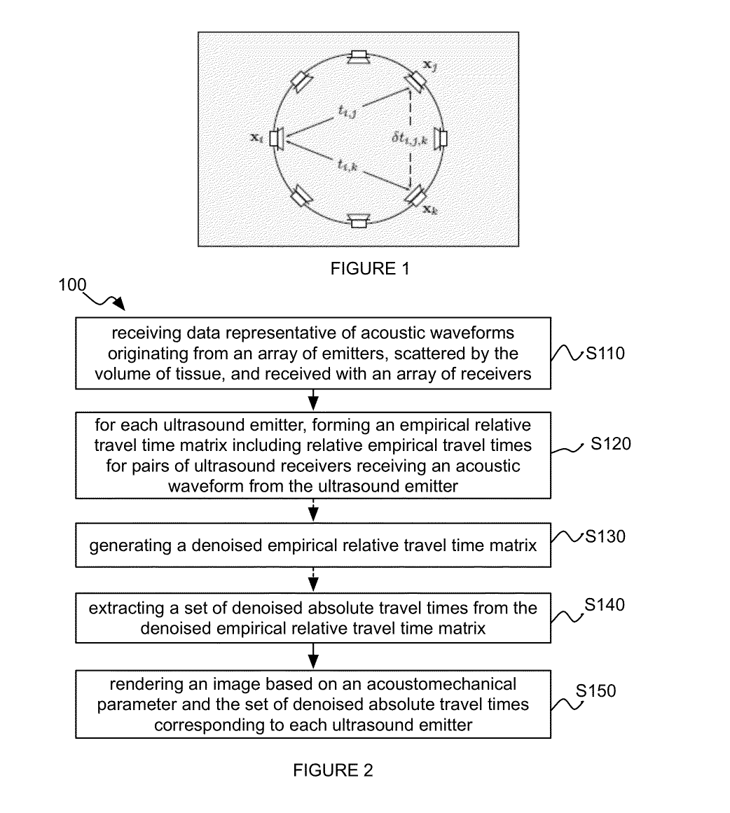

A minimization expression, derived using optimization theory, for denoising acoustic travel times is derived as follows: an example tomographic setup used in the derivation, as shown in FIG. 1, includes n ultrasound transducers with positions xᵢ (i=0, 1, . . . , n−1). The absolute travel time measured between transducers i and j is denoted as t_{i,j}, and the relative travel time between transducers j and k when a signal is emitted from transducer i is denoted as δt_{i,j,k}. For a given emitter i, one can stack all absolute travel times into a vector tᵢ, such that (tᵢ)ⱼ=t_{i,j}. A relative travel time matrix ΔTᵢ is formed such that (ΔTᵢ)_{j,k}=δt_{i,j,k}. It holds that

ΔTᵢ=tᵢ1^{T}−1tᵢ^{T} (1)

for i=0, 1, . . . , n−1. In Equation (1), the vector 1 denotes the all-one vector of size n. In the presence of noise, however, the equality of Equation (1) does not hold anymore. Therefore, an optimized set of denoised travel times is that which solves the minimization expression of

$$\text{?} {‖ {t}_{i} {1}^{T} - 1 {t}_{i}^{T} - {Δ}^{⋀} {T}_{i} ‖}^{2} \\ \text{?} \text{indicates text missing or illegible when filed} \qquad ( 2 )$$

where \hat{Δ}\hat{T}ᵢ denotes the noisy relative travel time measurements for emitter i. In the minimization expression of Equation (2), enforcement of the equality constraint t_{i,i}=0 prevents the system from having an infinite number of solutions. In this constraining case, an absolute travel time of zero is equivalent to a relative travel time where the emitter and the second receiver are the same (t_{i,j}=δt_{i,j,i}). Note that, if reciprocity holds (t_{i,j}=t_{j,i}), the travel times for different emitters can be optimized jointly using a similar formulation. The cost function in the minimization expression of Equation (2) can be rewritten as:

$$\begin{matrix}{‖ {t}_{i} {1}^{T} - 1 {t}_{i}^{T} - {Δ}^{⋀} {T}_{i} ‖}^{2} = {‖ vec ( {t}_{i} {1}^{T} - 1 {t}_{i}^{T} - {Δ}^{⋀} {T}_{i} ) ‖}^{2} \\ = {‖ {At}_{i} - {b}_{i} ‖}^{2} ,\end{matrix}$$

$$where$$

$$A = {[ 0 {C}_{1}^{T} … {C}_{n - 1}^{T} ]}^{T} and {b}_{i } = vec ( {Δ}^{⋀} {T}_{i} ) .$$

and where vec denotes the vec operator where the elements in the matrix are scanned circularly along the diagonals, starting with the main diagonal. The matrix o is the all-zero matrix of size n×n, and Cᵢ is the circulant matrix of size n×n whose first row has a one at indices 1 and i+1, and zero elsewhere. The minimization expression of Equation (2) can thus be expressed as

$$\text{?} {t}_{i} {A}^{T} {At}_{i} - 2 {b}^{T} {At}_{i} . \\ \text{?} \text{indicates text missing or illegible when filed} \qquad ( 3 )$$

Embodiments of a method 100 for denoising acoustic travel times, as presented below, comprise forming an empirical relative travel time matrix for each ultrasound emitter and, in several embodiments, denoising the empirical relative travel time matrix to form approximate solutions to the minimization expression (3), in order to extract denoised acoustic travel times.

### 2. Method for Denoising Acoustic Travel Times and Imaging a Volume of Tissue

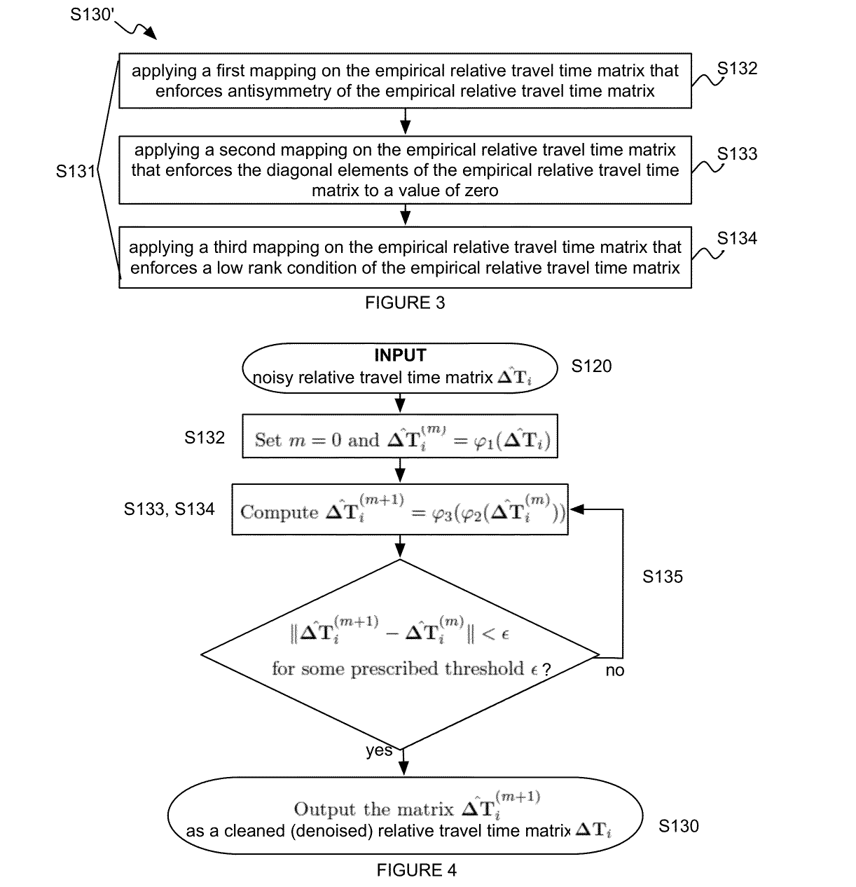

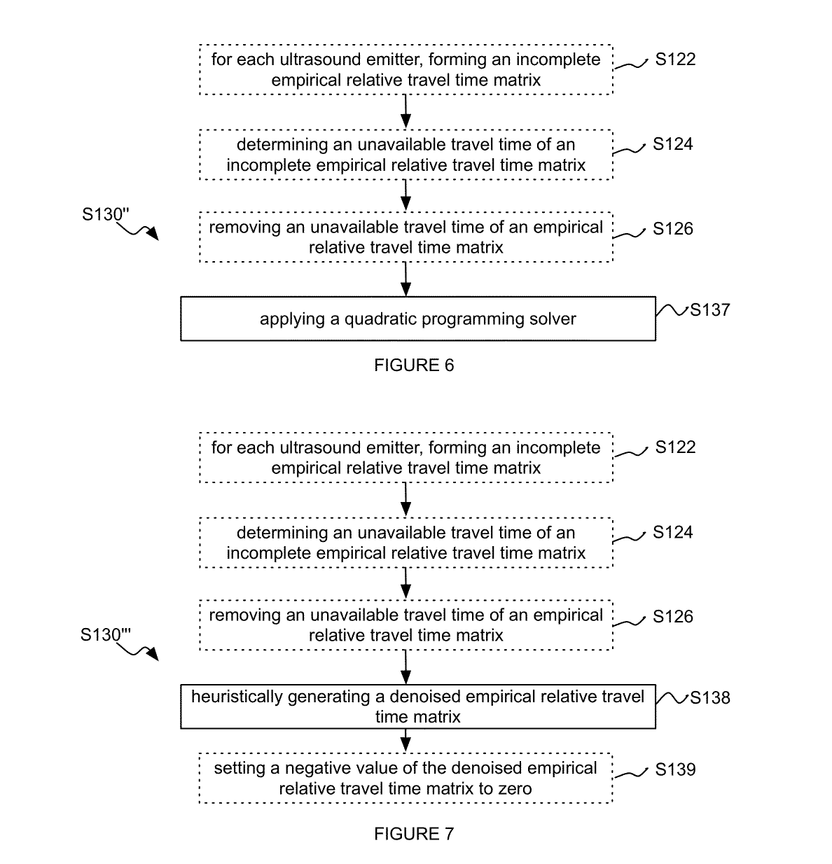

As shown in FIGS. 2 and 5, a method 100 for denoising acoustic travel times and imaging a volume of tissue includes: receiving a set of data representative of acoustic waveforms originating from an array of ultrasound emitters, scattered by the volume of tissue, and received with an array of ultrasound receivers surrounding the volume of tissue S110; for each ultrasound emitter in the array of ultrasound emitters, forming an empirical relative travel time matrix from the set of data S120, including a set of relative empirical travel times, generating a denoised empirical relative travel time matrix S130, and extracting a set of denoised absolute travel times from the denoised empirical relative travel time matrix S140, and rendering an image of the volume of tissue based on an acoustomechanical parameter and the set of denoised absolute travel times corresponding to each ultrasound emitter in the array of ultrasound emitters S150.

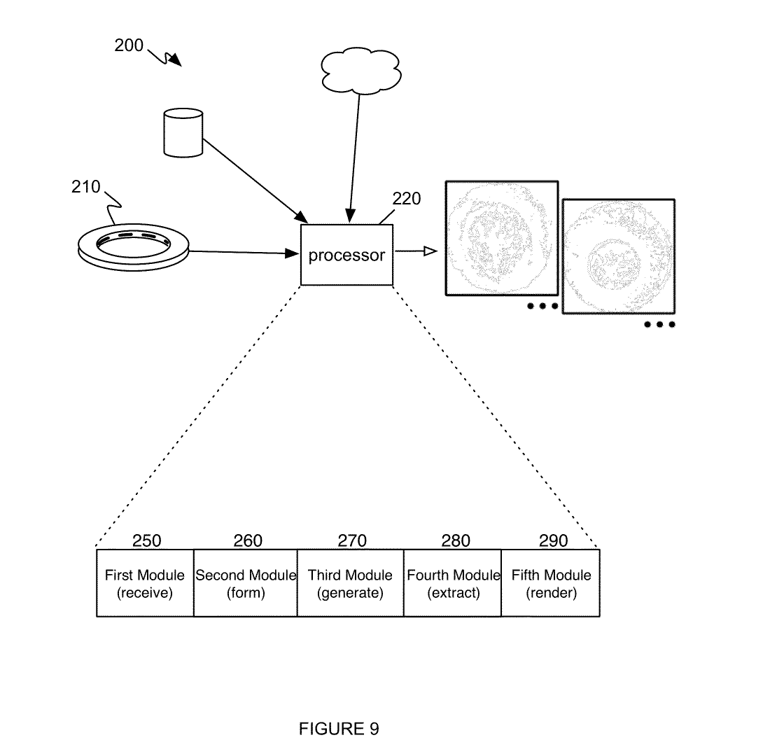

Step S110, which recites: receiving a set of data representative of acoustic waveforms originating from an array of ultrasound emitters, scattered by the volume of tissue, and received with an array of ultrasound receivers surrounding the volume of tissue, preferably functions to receive acoustic data as an information source from which acousto-mechanical characteristics of the volume of tissue can be derived. In a preferred embodiment, S110 includes receiving data directly from a transducer comprising a ring-based tomographic setup, similar to that shown in FIGS. 1, 8B, and 8C. In an alternative variation, S110 includes receiving data from a transducer comprising an alternative tomographic setup appropriate for the tissue volume for which an image is being rendered. In other alternative variations, S110 includes receiving data from a computer-readable medium or storage, such as a server, cloud storage, hard drive, flash memory, optical device (CD or DVD), or other suitable device capable of receiving, storing, and/or otherwise transferring acoustic data.

Step S120 recites: for each ultrasound emitter in the array of ultrasound emitters, forming an empirical relative travel time matrix from the set of data, including a set of relative empirical travel times, each relative empirical travel time corresponding to a pair of ultrasound receivers receiving an acoustic waveform. Step S120 preferably functions to organize a set of acoustic data into a format that facilitates processing, and to which a plurality of mappings can be applied. As an example using index notation, a relative empirical travel time for receivers j and k receiving an acoustic waveform emitted from ultrasound emitter i is preferably defined as the difference between the time of travel for a signal passing from emitter i to receiver j and the time of travel for a signal passing from emitter i to receiver k. For each ultrasound emitter in the array of ultrasound emitters, forming an empirical relative travel time matrix from the set of data S120 preferably functions to organize the acoustic data in a format to which a plurality of mappings can be applied. The empirical relative travel time matrix can also be expressed as a noisy and/or non-ideal relative time matrix \hat{Δ}Tᵢ, which may in some embodiments, be a product of cross-talk among nearby transducers, non-ideal frequency responses of sensors, and/or strong attenuation in a propagation medium.

As shown in FIG. 5, one variation of S120 can further include forming an incomplete empirical relative travel time matrix S122 corresponding to an ultrasound emitter in the array of ultrasound emitters or corresponding to each ultrasound emitter in the array of ultrasound emitters. Step S122 preferably functions to organize an incomplete set of acoustic data into a format that facilitates processing and to which a plurality of mappings can be applied. In some applications, it might not be possible to measure all the entries of the relative travel time matrix. For instance, the signals measured between some transducer pairs can be too noisy to provide relevant absolute travel time estimates. Noisy measurement can result, for example, if the incidence angle of the propagating acoustic wavefront is too large compared to the transducer beam width. The distortion incurred by a frequency dependent angular response also might have an adverse effect on the estimation of the travel time. Another potential reason for missing entries is the significant attenuation of an acoustic signal by the volume of tissue (e.g., dense breast tissue), thereby preventing a reasonable estimate of an absolute travel time. Relative travel time estimation between two signals (e.g., using a cross-correlation method) becomes challenging when the signals have different shapes.

In an embodiment of the method 100 comprising S122, which recites forming an incomplete empirical relative travel time matrix, the method may also further comprise S124, which recites: determining an unavailable travel time of the incomplete empirical relative travel time matrix. Determining an unavailable travel time of the incomplete empirical relative travel time matrix S124 preferably functions to fill in the missing entry or entries by interpolation. Determining an unavailable travel time of the incomplete empirical relative travel time matrix S122 preferably forms a patched empirical relative travel time matrix for further processing. As examples, S124 can include performing a suitable low-rank matrix completion algorithm, an interpolation technique based on geometrical considerations, any interpolation technique based on convex optimization, or any suitable interpolation algorithm. Alternatively, the method 100 may comprise removing an unavailable travel time or travel times in an incomplete empirical relative travel time matrix S126, as shown in FIG. 5.

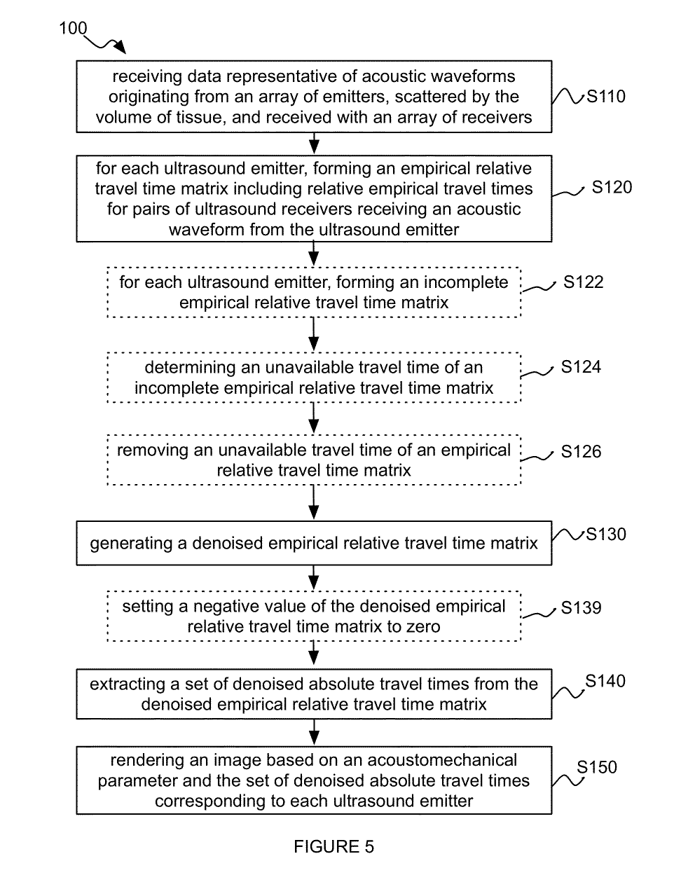

Step S130 recites: generating a denoised empirical relative travel time matrix, which preferably functions to iteratively process an empirical relative travel time matrix, such that it approximates an ideal (i.e. noiseless and/or complete) relative travel time matrix. Preferably, the denoised empirical relative travel time matrix optimally satisfies the minimization expression (3) derived in section 1 above but alternatively, the denoised empirical relative travel time matrix may approximately satisfy the minimization expression (3) derived in section 1. Embodiments where the denoised empirical relative travel time matrix approximately satisfies the minimization expression (3) include embodiments where the method functions, for example, to reduce computational resource expenditures. In the example, sub-optimal, but approximate solutions may be appropriate. In yet other alternative embodiments, the denoised empirical relative travel time matrix may also be generated based on any appropriate convex optimization techniques, computational methods for noise removal and/or optimization of data, and or any technique that functions to remove noise from a travel time data set. Sections 3.1-3.3 of the specification and FIGS. 3, 6, and 7 describe three embodiments of generating a denoised empirical relative travel time matrix S130.

Step S140 recites: for each ultrasound emitter in the array of ultrasound emitters, extracting a set of denoised absolute travel times from the denoised empirical relative travel time matrix. Step S140 preferably functions to obtain denoised travel times for use in acoustic tomography. In an embodiment, a denoised absolute travel time vector \hat{t}ᵢ for a given emitter i can be extracted from the ith column (or row, depending upon matrix layout) of the final, denoised empirical relative travel time matrix. The method 100 can further include applying a constraint of non-negativity to the empirical relative travel time matrix or separately to one or more of the extracted denoised absolute travel time vectors.

Step S150 recites: rendering an image of the volume of tissue based on an acoustomechanical parameter and the set of denoised absolute travel times corresponding to each ultrasound emitter in the array of ultrasound emitters, which preferably functions to provide an image of a volume of tissue for applications such as screening and/or diagnosis of cancer within the volume of tissue. As an example, S150 can be used to characterize regions of interest in the tissue (e.g., to characterize a suspicious mass as a tumor, a fibroadenoma, a cyst, another benign mass, and/or any suitable classification) or for monitoring status of the tissue such as throughout a cancer treatment. Preferably, S150 transforms the set of denoised absolute travel times from S140, into a rendered image that, for example, depicts a distribution of sound speed values within the scanned volume of tissue. Alternatively, the rendered image may depict a distribution of any appropriate acoustomechanical parameter, such as acoustic reflection or acoustic attenuation, or a combination of acoustomechanical parameter values, within the scanned volume of tissue. Rendering an image of the volume of tissue based on an acoustomechanical parameter and the set of denoised absolute travel times corresponding to each ultrasound emitter in the array of ultrasound emitters S150 preferably comprises rendering at least one two-dimensional image rendering representing the distribution of an acoustomechanical parameter (acoustic speed, acoustic reflection, acoustic attenuation, or combination of acoustomechanical parameters) within a cross-sectional plane of the scanned volume of tissue; however, S150 can additionally or alternatively comprise rendering a three-dimensional volumetric image representing an acoustomechanical parameter or combination of acoustomechanical parameters within the scanned volume of tissue. Methods of rendering an image are described in U.S. application Ser. No. 13/027,070 filed 14-FEB-2011 and entitled “Method of Characterizing Tissue of a Patient” which is incorporated in its entirety by this reference. A rendered image resulting from S150 may be displayed on a user interface, computer display, or any alternative display.

The FIGURES illustrate the architecture, functionality and operation of possible implementations of systems, methods and computer program products according to preferred embodiments, example configurations, and variations thereof. In this regard, each block in the flowchart or block diagrams may represent a module, segment, or portion of code, which comprises one or more executable instructions for implementing the specified logical function(s). It should also be noted that, in some alternative implementations, the functions noted in the block can occur out of the order noted in the FIGURES. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

### 3. Embodiments of S130: Generating a Denoised Empirical Relative Travel Time Matrix

In summary of Section 2, the method 100 for denoising acoustic travel times and imaging a volume of tissue includes: receiving a set of data representative of acoustic waveforms originating from an array of ultrasound emitters, scattered by the volume of tissue, and received with an array of ultrasound receivers surrounding the volume of tissue S110; for each ultrasound emitter in the array of ultrasound emitters, forming an empirical relative travel time matrix from the set of data S120, including a set of relative empirical travel times, generating a denoised empirical relative travel time matrix S130, and extracting a set of denoised absolute travel times from the denoised empirical relative travel time matrix S140; and rendering an image of the volume of tissue based on an acoustomechanical parameter and the set of denoised absolute travel times corresponding to each ultrasound emitter in the array of ultrasound emitters S150. As stated above, three embodiments of generating a denoised empirical relative travel time matrix S130, accompanied by embodiments of S140 and S150, are presented below in sections 3.1-3.3:

### 3.1 First Embodiment of Generating a Denoised Empirical Relative Travel Time Matrix

As shown in FIGS. 3 and 4, a first embodiment of generating a denoised empirical relative travel time matrix S130 preferably comprises applying a plurality of mappings to the empirical relative travel time matrix S131. Applying a plurality of mappings to the empirical relative travel time matrix S131 preferably functions to enforce at least one property of redundancy between absolute and relative time delays in the empirical relative travel time matrix, such that redundancy is used to denoise travel time data.

In a first embodiment of S131 the ideal or noiseless relative travel time matrix (1) is antisymmetric (that is, ΔTᵢ=−ΔTᵢ^{T}), (2) has diagonal elements of value zero, and (3) is of rank of at most 2. The first two properties are trivial and readily understood by one of ordinary skill in the art. The third property follows directly from the first property, since rank (tᵢ1^{T}−1tᵢ^{T})≦rank tᵢ1T^{T}+rank 1tᵢ^{T})≦2. The third property of low rank suggests that, in the noiseless case, the entries of the matrix ΔTᵢ are highly redundant. This redundancy is preferably used to denoise the travel time data in the empirical relative travel time matrix \hat{Δ}\hat{T}ᵢ. With noisy measurements, however, some of the above three properties might not be satisfied. The applied mappings preferably successively enforce these properties as a means to denoise the travel time data.

In the first embodiment of S131 the plurality of mappings preferably comprise at least three mappings that enforce the three properties of the relative time travel matrix. However, the plurality of mappings can additionally or alternatively include any suitable mappings that drive the empirical relative travel time matrix to have properties of an ideal, noiseless empirical relative travel time matrix. A first mapping φ₁, which enforces antisymmetry of the empirical relative travel time matrix, is preferably defined as φ₁(ΔTᵢ)=(ΔT₁−ΔTᵢ^{T})/2. A second mapping φ₂, which enforces the diagonal elements of the empirical relative travel time matrix to a value of zero, is preferably defined as (φ₂(ΔTᵢ))_{j,k}=(ΔTᵢ)_{j,k} if j≠k, and zero otherwise. A third mapping φ₃, which preferably enforces the low rank condition by retaining only the two largest singular values, is preferably defined as φ₃(ΔTᵢ)=U₂Λ₂V₂^{T}; that is, the best rank 2 approximation of ΔTᵢ using its singular value decomposition. As shown in FIG. 3, the first, second, and third mappings are preferably applied in blocks S132, S133, and S134, respectively.

As shown in FIG. 4, the method 100 may also further comprise repeating application of at least a portion of the plurality of mappings to an iteration of the empirical relative travel time matrix until a threshold is satisfied S135, thus generating the denoised empirical relative travel time matrix. Repeating application of at least a portion of the plurality of mappings to an iteration of the empirical relative travel time matrix until a threshold is satisfied S135 preferably functions to iteratively refine the empirical relative travel time matrix to a sufficiently denoised version. Preferably, in the first embodiment described above, S135 includes repeating application of the second mapping φ₂ and the third mapping φ₃; however, variations of S135 may include repeating application of the first mapping, the second mapping, the third mapping, and/or any additional appropriate mappings, in any appropriate application sequence. In the first embodiment, the antisymmetry condition imposed by the first mapping φ₁ is preferably not violated by the second and third mappings; therefore, the first mapping does not need to be repeated. Also in the first embodiment, the Frobenius norm of the empirical relative travel time matrix \hat{Δ}\hat{T}ᵢ is reduced at each iteration or successive repetition of the second and third mappings φ₂ and φ₃. Therefore, the Frobenius norm of \hat{Δ}\hat{T}ᵢ^{(m)} at iteration m preferably quantifies the amount of noise that has been removed from \hat{Δ}\hat{T}ᵢ after m iterations. The empirical relative travel time matrix \hat{Δ}\hat{T}ᵢ^{(m)} whose Frobenius normal satisfies a denoised threshold (e.g., a numerical quantity) can be considered a final empirical relative travel time matrix that is sufficiently denoised. The first embodiment of S131, S132, S133, S134, and S135 can also be expressed by the flowcharts depicted in FIGS. 3 and 4.

As shown in FIGS. 2 and 5, the method 100 comprises for each ultrasound emitter in the array of ultrasound emitters, extracting a set of denoised absolute travel times from the denoised empirical relative travel time matrix S140. Extracting a set of denoised absolute travel times from the denoised empirical relative travel time matrix S140 preferably functions to obtain denoised travel times for use in acoustic tomography. In the first embodiment, a denoised absolute travel time vector \hat{t}ᵢ for a given emitter i can be extracted from the ith column of the final, denoised empirical relative travel time matrix. The method 100 can further include applying a constraint of non-negativity to the empirical relative travel time matrix or separately to one or more of the extracted denoised absolute travel time vectors prior to rendering an image of the volume of tissue based on an acoustomechanical parameter and the set of denoised absolute travel times corresponding to each ultrasound emitter in the array of ultrasound emitters S150.

### 3.2 Second Embodiment of Generating a Denoised Empirical Relative Travel Time Matrix

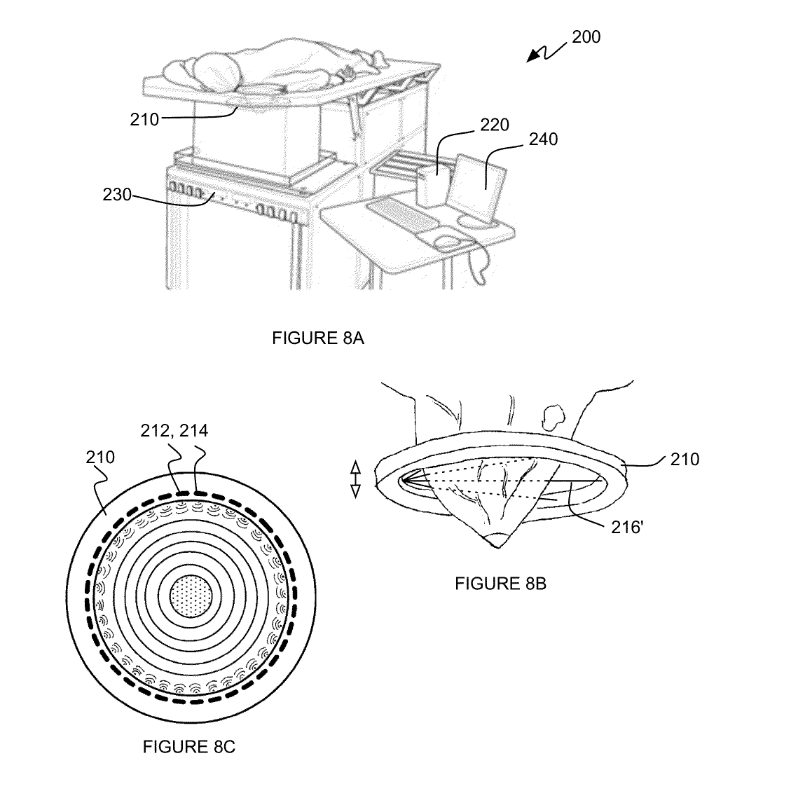

As shown in FIG. 6, a second embodiment of generating a denoised empirical relative travel time matrix S130 preferably comprises applying a quadratic programming solver S137 to generate a denoised empirical relative travel time matrix that approximately or ideally satisfies the minimization expression of Equation (3), which is reproduced below:

$$\text{?} {t}_{i} {A}^{T} {At}_{i} - 2 {b}^{T} {At}_{i} . \\ \text{?} \text{indicates text missing or illegible when filed} \qquad ( 3 )$$

In determining an analytical characterization of an approximation of the optimal solution of Equation (3), let C be the circulant matrix of size n×n with first row (n−1, −1, . . . , −1) and wᵢ the vector defined as wᵢ=ΔT [symbol] 1 with

$${Δ}^{⋀} {T}_{i} = \frac{1}{2} ( {Δ}^{⋀} {T}_{i} - {Δ}^{⋀} {T}_{i}^{T} ) . \qquad ( 4 )$$

The set ν is defined as the set of vectors v of the form v= C̅ [symbol] wᵢ, where C̅ contains a subset of the columns of the matrix C with indices j≠i. The unique solution of Equation (3) belongs to ν. This proposition is supported by the following:

The cost function of Equation (2) can be expressed as

$$\begin{matrix}{‖ {t}_{i} {1}^{T} - 1 {t}_{i}^{T} - {Δ}^{⋀} {T}_{i} ‖}^{2} = 2 n {‖ {t}_{i} ‖}^{2} - 2 tr ( {t}_{i}^{T} 1 {t}_{i}^{T} 1 ) + \\ tr ( {Δ}^{⋀} {T}_{i} {Δ}^{⋀} {T}_{i}^{T} ) - 2 tr ( {Δ}^{⋀} {T}_{i} ( 1 {t}_{i}^{T} - {t}_{i} {1}^{T} ) ) \\ = 2 n {‖ {t}_{i} ‖}^{2} - 2 tr ( {t}_{i}^{T} 1 {t}_{i}^{T} 1 ) + \\ tr ( {Δ}^{⋀} {T}_{i} {Δ}^{⋀} {T}_{i}^{T} ) - 4 tr ( {Δ}^{⋀} {T}_{i} 1 {t}_{i}^{T} ) ,\end{matrix}$$

wherein the second equality uses the fact that tr (A)=tr (A^{T}) and tr (AB)=tr (BA) for conforming matrices, and defines

$${Δ}^{⋀} {T}_{i} = \frac{1}{2} ( {Δ}^{⋀} {T}_{i} - {Δ}^{⋀} {T}_{i}^{T} ) .$$

Defining wᵢ=Δ [symbol] Tᵢ1, the minimization expression of Equation (2) in section 1 above can be rewritten as

$$\begin{matrix} \text{?} {‖ {t}_{i} {1}^{T} - 1 {t}_{i}^{T} - {Δ}^{⋀} {T}_{i} ‖}^{2} = \text{?} 2 n {‖ {t}_{i} ‖}^{2} - 2 {( {t}_{i}^{T} 1 )}^{2} - 4 {w}_{i}^{T} {t}_{i} \\ = \text{?} n {\sum }_{j = 0}^{n - 1} {{t}_{j}^{2} ( {\sum }_{j = 0}^{n - 1} {t}_{j} )}^{2} - 2 {\sum }_{j = 0}^{n - 1} {w}_{j} {t}_{j} .\end{matrix}$$

$$\text{?} \text{indicates text missing or illegible when filed}$$

The function f(tᵢ) can be defined as the above cost function, the function gⱼ(tᵢ) can be defined as gⱼ(tᵢ)=−t_{i,j}≦0 as the inequality constraints, and the function h(tᵢ) can be defined as h(tᵢ)=t_{i,i}=0 as the equality constraint. Since f and gⱼ are continuously differentiable, and h is affine, the Karush-Kuhn-Tucker conditions provide necessary and sufficient conditions for optimality. In particular, the stationarity condition

$$∇ f ( {\hat{t}}_{i} ) + {\sum }_{j \neq i} {μ}_{j} ∇ {g}_{j} ( {\hat{t}}_{i} ) + λ ∇ h ( {\hat{t}}_{i} ) = 0$$

implies that the multipliers μⱼ must satisfy

$${μ}_{j} = 2 ( n {\hat{t}}_{i , j} - \text{?} {\hat{t}}_{i , j} - {w}_{i , j} ) . \\ \text{?} \text{indicates text missing or illegible when filed}$$

The complementary slackness condition μⱼgⱼ(tᵢ)=0 evaluates as

$$( n {\hat{ t}}_{i , j} - \text{?} {\hat{t}}_{i , j} - {w}_{i , j} ) {\hat{t}}_{i , j} = 0.$$

$$\text{?} \text{indicates text missing or illegible when filed}$$

The solution \hat{t} thus satisfies

C\hat{t}ᵢ=wᵢ,

where C is the circulant matrix defined above.

Applying a quadratic programming solver S137 to generate a denoised empirical relative travel time matrix can comprise using any suitable computational solver (e.g., “quadprog” function in MATLAB®) to find an optimal solution or an approximation to the optimal solution of the convex quadratic function expressed in Equation (3).

### 3.3 Third Embodiment of Generating a Denoised Empirical Relative Travel Time Matrix

As shown in FIG. 7, a third embodiment of generating a denoised empirical relative travel time matrix S130 preferably comprises heuristically generating a denoised empirical relative travel time matrix S138 that ideally or approximately satisfies the minimization expression of Equation (3), which is reproduced below:

$$\text{?} {t}_{i} {A}^{T} {At}_{i} - 2 {b}^{T} {At}_{i} . \\ \text{?} \text{indicates text missing or illegible when filed} \qquad ( 3 )$$

Similar to the description of the second embodiment of generating a denoised empirical relative travel time matrix S130 described above in section 2.2, in determining an analytical characterization of an optimal solution of Equation (3), let C be the circulant matrix of size n×n with first row (n−1, −1, . . . , −1) and wᵢ the vector defined as wᵢ=Δ [symbol] Tᵢ1 with

$${Δ}^{⋀} {T}_{i} = \frac{1}{2} ( {Δ}^{⋀} {T}_{i} - {Δ}^{⋀} {T}_{i}^{T} ) . \qquad ( 4 )$$

However, in the third embodiment, the heuristic solution of Equation (3) is preferably defined as the vector v given by v= C̅ [symbol] wᵢ, where C̅ contains all of the columns (as opposed to a subset as in method 200) of the columns of the matrix C with indices j≠i. The multiplication by the pseudo-inverse C [symbol] can be efficiently implemented using a Fast Fourier Transform (FFT) or alternatively, any suitable computational solver. The third embodiment preferably includes setting the negative values of the solution ν to zero, but alternatively may not include setting negative values of the denoised empirical relative travel time matrix to zero S139.

The third embodiment preferably is non-iterative and requires a relatively low amount of computation power, yet can provide sufficient or even optimal results.

### 4. System for Denoising Acoustic Travel Times and Imaging a Volume of Tissue

As shown in FIGS. 8A and 8C, the system 200 of a preferred embodiment for denoising acoustic travel times and imaging a volume of tissue comprises: an array of ultrasound emitters 212 configured to surround the volume of tissue and emit acoustic waveforms toward the volume of tissue; an array of ultrasound receivers 214 configured to surround the volume of tissue and receive acoustic waveforms scattered by the volume of tissue; and a processor 220 comprising a first module 250, a second module 260, a third module 270, a fourth module 280, and a fifth module 290. The processes performed by the preferred processor 220 can include one or more actions described above with reference to the methods and variations thereof. As shown in FIG. 8A, the system 200 can further include a display 240 on which the acoustic data and/or generated image rendering can be displayed, such as to a medical practitioner and/or the patient.

The system 200 is preferably used to image a volume of tissue, such as breast tissue, for screening and/or diagnosis of cancer within the volume of tissue. In other applications, the system 200 can be used to characterize regions of interest in the tissue (e.g., to characterize suspicious masses as a tumor, a fibroadenoma, a cyst, another benign mass, or any suitable classification) or for monitoring status of the tissue such as throughout a cancer treatment. However, the system 200 can be used in any suitable application for imaging any suitable kind of tissue with ultrasound tomography.

The system 200 for imaging a volume of tissue preferably generates and/or uses denoised absolute acoustic travel times to generate an image rendering of a volume of tissue, scanned by the ultrasound emitters 212 and ultrasound receivers 214 surrounding the tissue, depicting the distribution of an acoustomechanical parameter within the volume of tissue. Use of denoised acoustic travel times results in image renderings that have fewer artifacts and more accurate acoustic speed values, thereby leading to more a clearer and more accurate depiction of the scanned volume of tissue, compared to image renderings based on noisy acoustic travel times.

As shown in FIG. 8C, the system 200 preferably includes an array of ultrasound emitters 212 and an array of ultrasound receivers 214. The array of ultrasound emitters 212 preferably functions to irradiate the volume of tissue with acoustic waveforms from multiple locations distributed around the volume of tissue. The array of ultrasound receivers 214 preferably functions to receive the acoustic waveforms, at least a portion of which are preferably scattered by the volume of tissue. The emitters 212 and receivers 214 can be piezoelectric or any suitable kind of ultrasound components.

In a preferred embodiment shown in FIG. 8A, the system 200 preferably includes a scanning apparatus including a transducer array 210 that includes the tissue-encircling arrays of emitters 212 and receivers 214 for scanning breast tissue of a patient. In one specific variation of the system 200, the transducer array 210 is of substantially elliptical or substantially circular dimensions, and preferably includes two hundred fifty six approximately evenly distributed ultrasound elements that each emits a fan beam of ultrasound signals toward the breast tissue and opposite end of the ring, and receives ultrasound signals scattered by the breast tissue (e.g., transmitted by and/or reflected by the tissue). In another variation of the system 200, the transducer array 210 includes 2048 evenly distributed ultrasound elements. However, the preferred system 200 can include any suitable number of ultrasound emitters 212 and ultrasound receivers 214 in any suitable geometric configuration.

As shown in FIG. 8A, the transducer array 210 is preferably paired with a patient table having an aperture, such that a patient lying prone stomach-side down on the patient table can pass her breast through the aperture. The patient table is preferably set up with a water bath, positioned beneath the patient table aperture, that receives the breast tissue and houses the ring transducer of the system 200. The transducer array 210, while surrounding the breast tissue, moves sequentially to a series of points along a vertical path in an anterior-posterior direction, scanning a two-dimensional cross-sectional image (e.g., coronal image) of the breast at each point, such that the received data can be used to generate a stack or series of two-dimensional images over the entire volume of tissue (and/or a three-dimensional volumetric image of the tissue). The water bath preferably functions to act as an acoustic coupling medium between the transducer array and the tissue, and to suspend the breast tissue (thereby reducing gravitational distortion of the tissue).

As shown in FIG. 8A, the system 200 can also include a controller 230 that functions to control the actions of the transducer ring 210. The controller 230 preferably functions to control the acoustic signals transmitted from the ultrasound emitters 212 (e.g., frequency of waveforms and frequency of activation of the ultrasound emitters), and/or the physical movements of the transducer array relative to the volume of tissue. In particular, the controller preferably controls motion of the transducer array 210, including dictating spacing between the scanning points at which the scanning occurs and the rate of travel between the scanning points.

As shown in FIGS. 8A and 9, the preferred system 200 can include a processor 220 that functions to determine a set of denoised acoustic travel times and generate an acoustic speed image rendering of the volume of tissue at least partially based on the set of denoised acoustic travel times. The processor 220 can receive acoustic data directly from the transducer array 210 as shown in FIG. 8A, or can receive stored acoustic data from a storage device (e.g., server, cloud storage) as shown in FIG. 9. The processor 220 preferably generates the set of denoised acoustic travel times and comprises several modules. In an embodiment of the system 200, the processor preferably comprises a first module 250 configured to receive a set of data obtained from the array of ultrasound receivers, a second module 260 configured to form an empirical relative travel time matrix corresponding to an ultrasound emitter in the array of ultrasound emitters, including a set of relative empirical travel times, each relative empirical travel time corresponding to a pair of ultrasound receivers receiving an acoustic waveform, a third module 270 configured to generate a denoised empirical relative travel time matrix, a fourth module 280 configured to extract a set of denoised travel times from the denoised empirical relative travel time matrix, and a fifth module 290 configured to render an image of the volume of tissue based on an acoustomechanical parameter and the set of denoised absolute travel times. The processor 220 preferably performs all or a portion of the method described above.

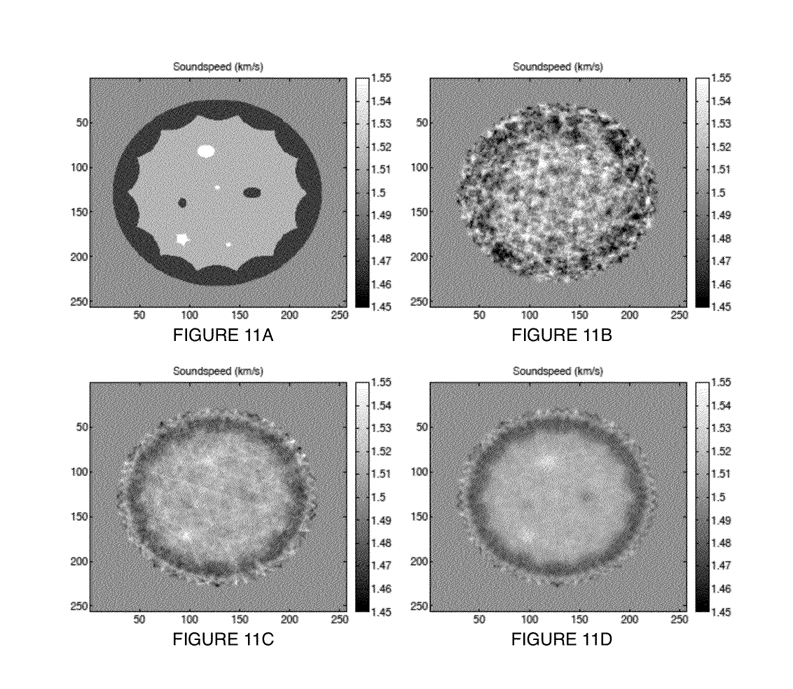

In an example implementation of the system 200 for denoising acoustic travel times and imaging a volume of tissue, a numerical sound speed phantom (shown in FIG. 11A) was imaged by an array of n=64 transducers. In the example implementation, a data set was generated using a time-domain waveform propagation scheme, and representative relative acoustic travel times were computed from estimated absolute travel times from the generated data. Additive white Gaussian noise is added to the relative travel times, to meet a desired experimental signal to noise ratio (SNR).

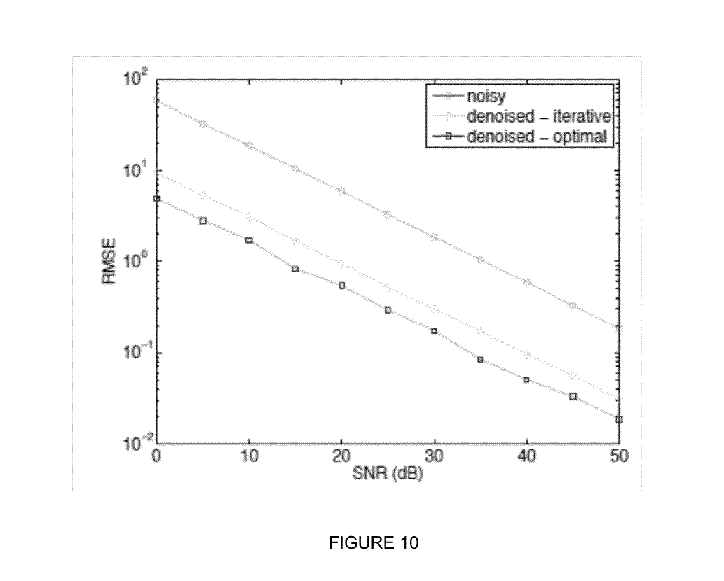

In the example implementation of the system 200, an iterative algorithm implemented by a first embodiment of the third module 270 applied repeated mappings to the a noisy relative travel time matrix including the noisy relative travel times and produced a denoised set of relative travel times. In another example implementation of the system 200, a quadratic programming solver implemented by a second embodiment of the third module 270 was used to denoise the noisy relative travel times in a mean-square optimal approach. In both example implementations of the system 200, respective sets of denoised absolute travel times were extracted from the denoised relative travel times. As shown in FIG. 10 (a plot of the root mean square error of travel times as a function of SNR), significant noise reduction is achieved by the two example implementations of the system 200.

As shown in FIG. 11B, a set of noisy acoustic travel times may be used to render a two-dimensional image of the numerical sound speed phantom. As shown in FIG. 11C, however, the system 200 may be used to render a two-dimensional image of the numerical sound speed phantom used in the above example implementations based on a set of acoustic travel times denoised using an iterative mapping approach (implemented by the third module 270). As shown in FIG. 11D, the system 200 may alternatively be used to render a two-dimensional image of the numerical sound speed phantom based on a set of acoustic travel times denoised using a mean-square optimal approach (implemented by the third module 270). FIGS. 11C and 11D thus suggest significant improvement in image quality compared to FIG. 11B, which was generated using noisy acoustic travel times, and not generated based on an embodiment of the system 200 for denoising acoustic travel times and imaging a volume of tissue.

The above example implementations of the system 200 are for illustrative purposes only, and should not be construed as definitive or limiting of the scope of the claimed invention.

The system and methods of the preferred embodiment and variations thereof can be embodied and/or implemented at least in part as machine configured to receive a computer-readable medium storing computer-readable instructions. The instructions are preferably executed by computer-executable components preferably integrated with the system and one or more portions of the processor 220 and/or the controller 230. The computer-readable medium can be stored on any suitable computer-readable media such as RAMs, ROMs, flash memory, EEPROMs, optical devices (CD or DVD), hard drives, floppy drives, or any suitable device. The computer-executable component is preferably a general or application specific processor, but any suitable dedicated hardware or hardware/firmware combination device can alternatively or additionally execute the instructions.

As a person skilled in the art will recognize from the previous detailed description and from the figures and claims, modifications and changes can be made to the preferred embodiments of the invention without departing from the scope of this invention defined in the following claims.

We claim:

1. A method for denoising acoustic travel times and imaging a volume of tissue comprising:
receiving a set of data representative of acoustic waveforms originating from an array of ultrasound emitters, scattered by the volume of tissue, and received with an array of ultrasound receivers;
for each ultrasound emitter in the array of ultrasound emitters,
forming an empirical relative travel time matrix from the set of data, including a set of relative empirical travel times, each relative empirical travel time corresponding to a pair of ultrasound receivers receiving an acoustic waveform,
generating a denoised empirical relative travel time matrix, and
extracting a set of denoised absolute travel times from the denoised empirical relative travel time matrix; and
rendering an image of the volume of tissue based on an acoustomechanical parameter and the set of denoised absolute travel times corresponding to each ultrasound emitter in the array of ultrasound emitters.

2. The method of claim 1, wherein receiving a set of data representative of acoustic waveforms originating from an array of ultrasound emitters, scattered by the volume of tissue, and received with an array of ultrasound receivers comprises receiving a set of data from a ring-shaped ultrasound transducer.

3. The method of claim 1, wherein for each ultrasound emitter in the array of ultrasound emitters, generating a denoised empirical relative travel time matrix comprises generating a denoised empirical relative travel time matrix based on an optimization technique.

4. The method of claim 1, wherein for each ultrasound emitter in the array of ultrasound emitters, generating a denoised empirical relative travel time matrix comprises:
applying a plurality of mappings to the empirical relative travel time matrix; and
repeating application of at least a portion of the plurality of mappings to an iteration of the empirical relative travel time matrix until a threshold is satisfied, thus generating the denoised empirical relative travel time matrix.

5. The method of claim 4, wherein applying a plurality of mappings includes reinforcing a property of redundancy between absolute and relative time delays.

6. The method of claim 4, wherein for each ultrasound emitter in the array of ultrasound emitters, applying a plurality of mappings to the empirical relative travel time matrix comprises applying at least one of:
a first mapping, that characteristically enforces matrix antisymmetry, to the empirical relative travel time matrix;
a second mapping, that forces diagonal elements a matrix to a value of zero, to the empirical relative travel time matrix; and
a third mapping, that enforces a rank 2 condition using a singular value decomposition, to the empirical relative travel time matrix.

7. The method of claim 6, wherein applying a plurality of mappings to the empirical relative travel time matrix comprises applying the first mapping, applying the second mapping, and applying the third mapping in succession.

8. The method of claim 6, wherein for each ultrasound emitter in the array of ultrasound emitters, repeating application of at least a portion of the plurality of mappings to an iteration of the empirical relative travel time matrix comprises repeating application of the second mapping and the third mapping.

9. The method of claim 6, wherein for each ultrasound emitter in the array of ultrasound emitters, repeating application of at least a portion of the plurality of mappings to an iteration of the empirical relative travel time matrix until a threshold is satisfied comprises comparing a norm of an expression containing several iterations of the empirical relative travel time matrix to the threshold.

10. The method claim 4, wherein for each ultrasound emitter in the array of ultrasound emitters, forming an empirical relative travel time matrix comprises forming an incomplete empirical relative travel time matrix.

11. The method of claim 10, wherein for each ultrasound emitter in the array of ultrasound emitters, generating a denoised empirical relative travel time matrix based on a solution to a minimization problem comprises:
determining an unavailable travel time of the incomplete empirical relative travel time matrix based on interpolation, thus forming a patched empirical relative travel time matrix;
applying a plurality of mappings to the patched empirical relative travel time matrix; and
repeating application of at least a portion of the plurality of mappings to an iteration of the patched empirical relative travel time matrix until a threshold is satisfied, thus generating the denoised empirical relative travel time matrix.

12. The method of claim 11, wherein for each ultrasound emitter in the array of ultrasound emitters, determining an unavailable travel time of the incomplete empirical relative travel time matrix based on interpolation comprises using a low-rank matrix completion algorithm.

13. The method of claim 11, wherein for each ultrasound emitter in the array of ultrasound emitters, determining an unavailable travel time of the incomplete empirical relative travel time matrix based on interpolation comprises using an interpolation technique based on a geometrical consideration.

14. The method of claim 1, wherein for each ultrasound emitter in the array of ultrasound emitters, generating a denoised empirical relative travel time matrix comprises applying a quadratic programming solver.

15. The method of claim 14, wherein generating a denoised empirical relative travel time matrix based comprises removing any unavailable relative travel time values from the empirical relative travel time matrix prior to applying the quadratic programming solver.

16. The method of claim 1, wherein for each ultrasound emitter in the array of ultrasound emitters, generating a denoised empirical relative travel time matrix comprises heuristically generating a denoised empirical relative travel time matrix.

17. The method of claim 16, further comprising setting any negative values of the denoised empirical relative travel time matrix to zero.

18. The method of claim 1, wherein rendering an image of the volume of tissue based on an acoustomechanical parameter and the set of denoised absolute travel times corresponding to each ultrasound emitter in the array of ultrasound emitters comprises rendering an acoustic speed image of the volume of tissue.

19. A method for denoising acoustic travel times and imaging a volume of tissue comprising:
receiving a set of data representative of acoustic waveforms originating from an array of ultrasound emitters, scattered by the volume of tissue, and received with an array of ultrasound receivers;
for each ultrasound emitter in the array of ultrasound emitters,
forming an empirical relative travel time matrix from the set of data, including a set of relative empirical travel times, each relative empirical travel time corresponding to a pair of ultrasound receivers receiving an acoustic waveform,
applying a plurality of mappings to the empirical relative travel time matrix,
repeating application of at least a portion of the plurality of mappings to an iteration of the empirical relative travel time matrix until a threshold is satisfied, thus generating a denoised empirical relative travel time matrix, and
extracting a set of denoised absolute travel times from the denoised empirical relative travel time matrix; and
rendering an image of the volume of tissue based on an acoustomechanical parameter and the set of denoised absolute travel times corresponding to each ultrasound emitter in the array of ultrasound emitters.

20. The method of claim 19, wherein for each ultrasound emitter in the array of ultrasound emitters, applying a plurality of mappings to the empirical relative travel time matrix comprises applying at least one of:
a first mapping, that characteristically enforces matrix antisymmetry, to the empirical relative travel time matrix;
a second mapping, that forces diagonal elements a matrix to a value of zero, to the empirical relative travel time matrix; and
a third mapping, that enforces a rank 2 condition using a singular value decomposition, to the empirical relative travel time matrix.

21. A system for denoising acoustic travel times and imaging a volume of tissue comprising:
an array of ultrasound emitters configured to surround the volume of tissue and emit acoustic waveforms toward the volume of tissue;
an array of ultrasound receivers configured to surround the volume of tissue and receive acoustic waveforms scattered by the volume of tissue; and
a processor comprising:
a first module configured to receive a set of data obtained from the array of ultrasound receivers,
a second module configured to form an empirical relative travel time matrix, corresponding to an ultrasound emitter in the array of ultrasound emitters, including a set of relative empirical travel times, each relative empirical travel time corresponding to a pair of ultrasound receivers receiving an acoustic waveform,
a third module configured to generate a denoised empirical relative travel time matrix, corresponding to the ultrasound emitter in the array of ultrasound emitters,
a fourth module configured to extract a set of denoised absolute travel times from the denoised empirical relative travel time matrix corresponding to the ultrasound emitter in the array of ultrasound emitters, and
a fifth module configured to render an image of the volume of tissue based on an acoustomechanical parameter and the set of denoised absolute travel times.

22. The system of claim 21, further comprising a ring transducer that houses the array of ultrasound emitters and array of ultrasound receivers.

23. The system of claim 21, wherein the third module is configured to generate a denoised empirical relative travel time matrix by applying a plurality of mappings to the empirical relative travel time matrix and repeating application of at least a portion of the plurality of mappings to an iteration of the empirical relative travel time matrix until a threshold is satisfied, thus generating the denoised empirical relative travel time matrix.

24. The system of claim 21, wherein the third module comprises a quadratic programming solver configured to generate a denoised empirical relative travel time matrix.

25. The system of claim 21, wherein the third module comprises a heuristic solver configured to generate a denoised empirical relative travel time matrix.
